(19) United States
(12) Patent Application Publication
(10) Pub. No.: US 2006/0039865 A1
(43) Pub. Date: Feb. 23, 2006

(54) Pharmaceutical compositions and methods for the prevention of drug misuse

(76) Inventors: David M. Preston, Chapel Hill, NC (US); James W. Newman, Rancho Santa Fe, CA (US); Matthew T. Heck, Encinitas, CA (US)

Correspondence Address: HUTCHISON & MASON PLLC, PO BOX 31686, RALEIGH, NC, 27612, US

(21) Appl. No.: 11/188,574

(22) Filed: Jul. 25, 2005

Related U.S. Application Data
(63) Continuation-in-part of application No. 10/899,213, filed on Jul. 26, 2004.

(51) Int. Cl. A61K 49/00

(52) U.S. Cl. 424/10.4

(57) ABSTRACT

A pharmaceutical composition including a therapeutic does of an active ingredient susceptible of either intentional or accidental misuse by over-utilization and a sub-therapeutic dose of a compound in an amount sufficient to elicit an undesirable response if the recommended therapeutic dose of the active ingredient is exceeded. A multiple dose pharmaceutical dosage form including an effective amount of an active ingredient for a patient in need of treatment thereof and a urine indicator capable of changing the color of the patient's urine when a desired dosage of the active ingredient is exceeded by ingestion of multiple doses in excess of a desired treatment regimen.

This application is a continuation in part application of U.S. application Ser. No. 10/899,213 filed Jul. 26, 2004, incorporated herein by reference in its entirety.

### FIELD

The compositions and methods disclosed herein generally relate to reducing or preventing the likelihood of substance misuse.

### BACKGROUND

There are a number of prescription and over-the-counter medications that are regularly misused by users ingesting amounts far in excess of the recommended dosages. These medications are also often stored in large quantities at home where children have easy access. Examples include active ingredients such as dextromethorphan found in many flu, cold, and cold and cough medicines. There are over 125 over-the-counter medications including dextromethorphan (DXM). Narcotics, sedative, hypnotics and other controlled substances are also frequently misused.

Misuse of DXM and other prescription and over-the-counter active ingredients is reportedly a significant problem. For example, DXM has become a drug of choice for teenagers because it is relatively inexpensive and readily accessible. It is difficult to control access to DXM is because it is an active ingredient in many medications found in supermarkets, drugstores, and convenience stores. Some sellers have voluntarily placed these items behind the counter or labeled them with anti-theft devices. Manufacturers have posted warnings on their web sites concerning the risk of misuse or abuse. These efforts are largely ineffective in addressing the problem.

U.S. Pat. No. 4,552,035 discloses a composition for inducing smoking cessation including tobacco and alkaloids from Radix ipecauanhae. A derivative of ipecac was mixed with tobacco to induce nausea in smokers thus reducing the desire to smoke.

U.S. Pat. No. 6,228,863 discloses a method of reducing the abuse potential of an oral dosage form of an opioid analgesic, wherein an analgesically effective amount of an orally active opioid agonist is combined with an opioid antagonist into an oral dosage form which would require at least a two-step extraction process to be separated from the opioid agonist, the amount of opioid antagonist including being sufficient to counteract opioid effects if extracted together with the opioid agonist and administered parenterally.

Lomotil, a commercially available combination of diphenoxylate HCl with atropine sulphate, is used for the treatment of diarrhea. Each tablet contains 2.5 mg diphenoxylate and 0.025 mg atropine—a ratio of 100 to 1 by weight. Diphenoxylate is a constipating meperiding congener that lacks analgesic activity but high doses may cause opioid activity. Both diphenoxylate and atropine slow bowel action. Atropine may also cause tachycardia. A product label for adult usage indicates a dosage of two 2.5 mg tablets per day and states that the dose which produces antidiarrheal action is widely separated from the dose which casues central nervous system effects. 100 to 300 mg or 40 to 120 tablets per day produced opiate withdrawal. At such doses, a patient would receive a minimum dosage of 2.5 mg of atropine—an amount that could cause slowing of bowel action or tachycardia.

A label for pediatric use states that a subtherapeutic dose of atropine may discourage deliberate abuse.

There are a number of medications taken on a routine or multi-dose basis that may present a risk of abuse. Some medications, particularly those taken in frequent daily doses such as analgesics, are difficult to keep track of in terms of the total dose ingested by a patient in a given day. As a result, desired dosages may be intentionally or inadvertently exceeded. It would, therefore, be desirable for a patient, or someone monitoring a patient, to be able to readily identify whether or not the total desired dose is exceeded without the necessity of a blood test to determine the concentration of drug in the bloodstream.

### SUMMARY

Pharmaceutical compositions and methods are described including a therapeutic dose of an active ingredient susceptible of misuse and a sub-therapeutic dose of an anticholinergic compound capable of crossing the blood-brain barrier in an amount sufficient to elicit an undesirable central nervous system (CNS) response in a patient exceeding the prescribed therapeutic dose of the active ingredient, wherein the ratio of active ingredient to anticholinergic is present in the ratio of 30,000:1 to 250:1 by weight. Such compositions provide the desired CNS response in instances of misuse while reducing the risk of slowing bowel movement or tachycardia which may cause serious complications in, for example, senior patient populations.

Compositions and methods disclosed herein may include a combination of an active ingredient and any non-anticholinergic compound capable of eliciting an undesirable response wherein the active ingredient and the compound capable of eliciting an undesirable response are present in a dosage form in a proportion such that the undesirable response is elicited when the recommended dose of the active ingredient is exceeded.

Further disclosed is a multiple dose pharmaceutical dosage form comprising an effective amount of an active ingredient for a patient in need of treatment thereof and a urine indicator capable of changing the color of the patient's urine when a desired dosage of the active ingredient is exceeded by ingestion of multiple doses in excess of a desired treatment regimen.

Also disclosed is a method for indicating when a patient ingesting multiple doses of a pharmaceutical dosage form exceeds a desired treatment regimen comprising:
a. observing the color of a patients urine;
b. ingesting one or more doses of a pharmaceutical dosage form including an effective amount of an active ingredient for a patient in need of treatment thereof and a urine indicator capable of changing the color of the patients urine when a desired dosage of the active ingredient is exceeded by ingestion of multiple doses in excess of a desired treatment regimen;
c. observing the color of a patient's urine after ingesting one or more of the doses;
d. comparing the color of the patent's urine before and after ingesting one or more of the doses to determine if there is a substantial change in color of the patient's urine.

### BRIEF DESCRIPTION OF THE DRAWING FIGURES

FIG. 1 demonstrates the results of overdosing compositions described herein.

FIG. 2(a), a table entitled “Gastrointestinal Anticholinergic/Antispasmodic Dosage”, lists examples of therapeutic and sub-therapeutic doses of various compounds capable of eliciting undesirable responses.

FIG. 2(b) lists daily therapeutic and daily subtherapeutic dosages of various anticholinergics.

FIG. 3. is a table listing examples of abused pharmaceuticals with sub-therapeutic agents.

### DETAILED DESCRIPTION

a. Combinations of Anticholinergics and Active Ingredients

Anticholinergics are a class of drugs that competitively antagonize muscarinic receptors. Muscarinic receptors are found, for example, in the cells of the heart, salivary glands, sweat glands, GI tract and GU tract. Some anticholinergics have Central Nervous System (CNS) activity resulting from central cortical and subcortical muscarinic receptor antagonism. The extent of CNS activity will depend upon a drug's ability to cross the blood-brain barrier. Anticholinergics have undesirable and uncomfortable side effects when taken in typical therapeutic quantities. However, they are relatively benign when taken in sub-therapeutic doses. Accordingly, a typical patient ingesting a therapeutic amount of an active pharmaceutical ingredient combined with a sub-therapeutic dose of an anticholinergic will not experience any of the undesirable effects of the anticholinergic. However, a patient that intentionally overdoses the active ingredient will experience undesirable effects of the anticholinergic as the effective dose of the anticholinergic reaches therapeutic ranges.

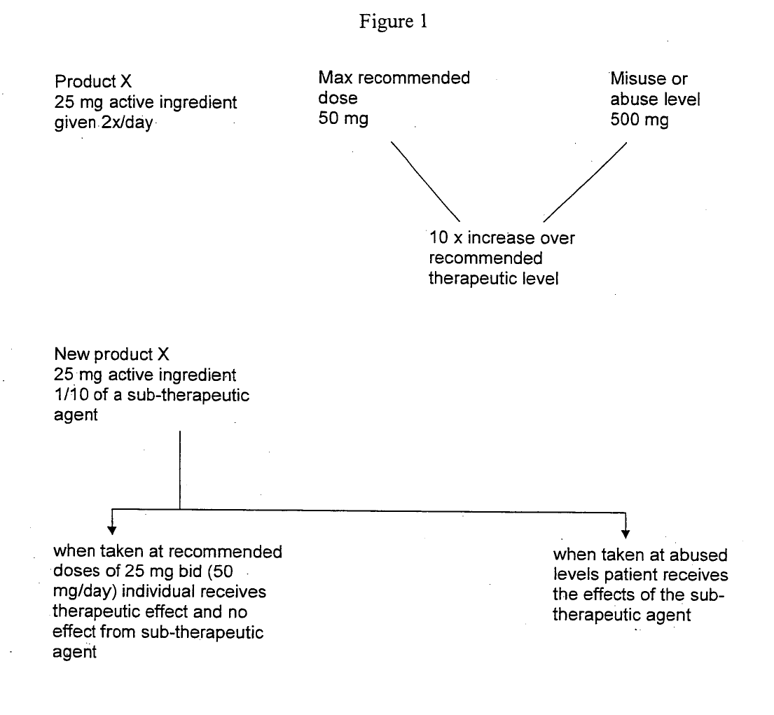

For example, a patient ingesting a combination liquid product including a single therapeutic dose of dextromethorphan per 10 ml of liquid product and a sub-therapeutic dose of atropine per 10 ml would not experience the undesirable effects of atropine. However if that same patient ingested 50 to 100 ml of the liquid product in a single dose, the patient may experience the physically uncomfortable effects of atropine explained below. See FIG. 1.

Atropine is an anticholinergic. It is useful in controlling heart function, treating diarrhea, excessive salivation, inducing dilation for eye exams and as a premedication for anesthesia and decreases bronchial and salivary secretions. At therapeutic doses, atropine will elicit undesirable responses including blurred vision, dryness of the mouth, photophobia, urinary retention, dizziness, and pupilary dilation.

There are many pharmaceutical agents, plants and chemicals that have anticholinergic properties. However, none are capable of crossing the blood-brain barrier to provide CNS activity. Examples of anticholinergics capable of crossing the blood-brain barrier include atropine, scopolamine, hyoscamine and belladonna alkaloids.

b. Other Combinations

Other non-anticholinergic compositions capable of eliciting an undesirable response include any agent with the ability to elicit undesirable side effects at therapeutic dosage ranges.

For example, undesirable responses include gastrointestinal responses (e.g., nausea vomiting, constipation, spacicity, bloating, reflux, flatulence, weight gain or weight loss), CNS related effects (e.g., drowsiness, dizziness, headache, sedation, agitation, insomnia, orthostatic hypotension), ocular effects (e.g., pupil dilation, pupil constriction, blurred vision), general malaise, dermatologic effects (e.g., rash, flushing), peripheral effects (e.g., tingling of extremities, numbness, excessive sweating, myalgia, arthralgia, alopecia, halitosis, drymouth), and genito-urinary effects (e.g., sexual dysfunction, urinary pigment changes, urinary retention). As mentioned above, anticholinergics may elicit CNS and ocular effects such as dizziness, blurred vision and papillary dilation. Antihistamines elicit CNS effects. Laxatives and ipecac elicit GI effects. Acarbose elicits flatulence and cyproheptadine may elicit weight gain. Hydralazine, nitroglycerin, and other vasodialators elicit orthostatic hypotension, dizziness, and headache. Caffeine elicits agitation. Undesirable peripheral responses may be elicited by vasodilators, nitroglycerin, and hydralazine which cause tingling and headache as well as pentosan polysulfate sodium which elicits alopecia. Dermatologic effects may be elicited by niacin which causes flushing. Genito-urinary effects may be elicited by guanethidine (sexual dysfunction), pyridium (changes to pigment of urine) and anticholinergics which elicit urinary retention.

Other examples of compositions capable of eliciting undesirable responses include those that would result in a lack of effect or blunted effect of the active agent due to the activity of the sub-therapeutic agent at the higher misused dosage level. This could occur, for example, through an effect of GI absorption of the active agent. For example, the sub-therapeutic agent could be an agent that binds the active ingredient in the GI tract and does not allow penetration such as activated charcoal or other binding substance. An example of such a composition is a combination propoxyphene napsylate with a sub-therapeutic dose of activate charcoal. Other examples include a combination of an active ingredient with a sub-therapeutic dose of composition that deactivates the method of action of the active ingredient (e.g. the composition capable of eliciting an undesirable response is an agent with a known drug-drug interaction that would negate or blunt the intended action of the active ingredient). An example is the combination of diazepam with a sub-therapeutic dose of phenytoin which would inhibit the metabolization of diazepam to its active metabolite.

Other examples of compounds capable of eliciting undesirable responses include compounds capable of negating the effect of the active agent when combined with a sub-therapeutic dose of an ingredient that would provide the opposite effect of the active ingredient. The sub-therapeutic dose of a compound capable of eliciting an undesirable response would counteract the effect of the active agent when misuse occurs. For example, a CNS depressant such as diazepam could be combined with a sub-therapeutic dose of a CNS stimulant such as caffeine. Another example is a combination of sibutramin with a sub-therapeutic dose of an antihistamine.

More than one compound capable of eliciting an undesirable response may be used in combination with one or more active ingredients.

Products and methods as described herein could include single or multiple active ingredients in a single composition or formulation. Misuse of an active ingredient is the ingestion of a dosage in excess of a recommended dose or frequency of administration. Misuse includes any accidental or intentional ingestion of an amount in excess of a recommended dose, frequency of dosing, or dosing regimen. Misuse also includes abuse.

c. Urine Color Indicators Combined With Active Ingredients

It would be advantageous for a patient, or someone monitoring a patient, to be able to observe a urine color change (shift) when a maximum dose of an active ingredient is exceeded. In this regard, it is desirable to combine a biocompatible substance capable of altering the color of a patient's urine (an indicator) with an effective amount of an active ingredient, e.g., acetaminophen. For example, each dosage form, e.g. tablet, capsule or suspension, may contain an amount of indictor that would not elicit a noticeable color shift unless multiple dosage forms were ingested effectively delivering a total dosage in excess of a desired amount.

This may be accomplished by a multiple dose pharmaceutical dosage form comprising an effective amount of an active ingredient for a patient in need of treatment thereof and a urine indicator capable of changing the color of the patient's urine when a desired dosage of the active ingredient is exceeded by ingestion of multiple doses in excess of a desired treatment regimen.

A multiple dose pharmaceutical dosage form is, for example, a tablet, capsule, liquid, or suspension containing an amount of active ingredient that is typically ingested in multiple time intervals with dosing regimens of one per day, twice daily, three times per day, four times per day, or more. It is often difficult for a patient to remember exactly how many doses of a particular medication were ingested during a given period. For example, anti-inflammatory drugs may be administered many times per day. A patient may have unknowingly exceeded the maximum desired dose for a given time period.

An effective amount of an active ingredient is an amount that will provide the desired medicinal effect. Active ingredients include those listed herein as active ingredients susceptible to misuse.

A urine indicator is any biocompatible composition that will change the color of a patient's urine upon ingestion. Indicators should be used in an amount such that they will change the color of a patient's urine when the desired treatment regimen is exceeded. For example, a dosage form that is taken four times per day should include an amount of indicator that will not substantially change the color of a patient's urine until five or more doses of the dosage form are taken. Alternatively, it may be desirable for a patient's urine to change color upon the taking of the last dose in a regimen. Various modifications of this will be apparent to those of skill in the art. Urine indicators that do not have serious side effects are preferred. Typical amount of indicator will vary depending upon the identity of the indicator but may be in the range of 5 mg to 750 mg per dosage form. For example, methylene blue may be used in an amount of 5 to 450 mg per tablet.

In determining the amounts of active ingredient and urine indicator to use in a given pharmaceutical dosage form, the total maximum daily desired dosage of the active and the amount of urine indicator necessary to cause a noticeable difference in pigment may be divided by the number of daily doses to give the amounts of each ingredient that should be included in each dosage form. For example, if a maximum dose of 4 mg per day of acetaminophen is desired and combined daily dosage of 300 mg of pyridium (phenazopyridine HCl) will elicit a noticeable change in urine color, each tablet should include 500 mg of acetaminophen and 40 mg of pyridim if up to eight tablets are to be ingested per day.

Various methods may incorporate the foregoing composition including a method for indicating when a patient ingesting multiple doses of a pharmaceutical dosage form exceeds a desired treatment regimen comprising:
a. observing the color of a patient's urine;
b. ingesting one or more doses of a pharmaceutical dosage form including an effective amount of an active ingredient for a patient in need of treatment thereof and a urine indicator capable of changing the color of the patient's urine when a desired dosage of the active ingredient is exceeded by ingestion of multiple doses in excess of a desired treatment regimen;
c. observing the color of a patient's urine after ingesting one or more of the doses;
d. comparing the color of the patent's urine before and after ingesting one or more of the doses to determine if there is a substantial change in color of the patient's urine.

Observing the color of a patient's urine before and after ingesting one or more doses of a pharmaceutical dosage form may accomplished by a variety of methods known to those of ordinary skill in the art. For example, a specimen may be collected in a glass vial, or on an absorbent test strip. The sample may then be matched to the closest color on a color spectrum chart. Alternatively, a patient may simply monitor its urine color before and after a dosing regimen. More quantitative methods may make use of a spectrophotometer to detect color.

Comparing the color of the patient's urine before and after ingesting one or more doses of a pharmaceutical dosage form may be accomplished, for example, by comparing the wavelength of light associated with each sample or comparing the closest matches on the color spectrum chart to determine if there has been a substantial change in color of the patient's urine. It may be desirable to take samples at the same time of day. For example, morning urine will naturally be darker in color than urine taken after eating. Similarly, ingestion of large amounts of liquids may impact urine color. There are also drugs and medical conditions that may elicit changes in urine color. Accordingly, it may be desirable to note whether or not the actual color change is consistent with the expected color change based upon the identity of the particular indicator selected.

A substantial change in the color of a patient's urine may occur when the naked eye is able to discern a difference. A substantial change in color may also occur when a change of 10 nanometers (nm) or more is observed between the sample taken before and after dosing the patient.

The following list of urine indicators (and all pharmaceutically acceptable salts thereof) is included by way of example:

| INDICATOR | COLOR |
|---|---|
| Blutene | blue-green |
| Eosin dyes(H) | brown, red, pink |
| Ethoxazene | orange, red, orange-red, pink |
| Evans blue | blue |
| Ferrous salts | black, dark |
| Mannose | orange |
| Melanin(OS) | black, dark |
| Methylene blue | blue, green, blue-green, yellow-brown, |
| | green-yellow |
| Phenazopyridine(AC) | orange-red, red, orange |
| Phenolphthalein(AL) | pink, red, red-brown |
| Phenolsulfonphthalein(AL) | red, red-purple |
| Phenazopyridine | orange |
| Riboflavin | dark, rust, orange, orange-yellow, red, |
| | brown, yellow |
| Tannins | brown |

d. Active Ingredients Susceptible to Misuse

Active ingredients susceptible to misuse include any pharmaceutical compound, prescription compound, or over the counter product with the potential for misuse through exceeding recommended dosages or misuse through overutilizing either intentional or accidental. Examples include: (1) any CII-CV controlled substances, (2) any non-controlled stimulant like agents, for example, pseudo-ephedrine or ephedrine, (3) any non-controlled cough suppressent or analgesic with narcotic type effect, for example dextromethorphan, (4) any non-controlled sedative hypnotic type agents, (5) any “quality of life enhancing” agents where overutilization can be hazardous, for example, appetite suppressant, weight control agents, and erectile dysfunction agents, and (6) any therapeutic agent with a known “therapeutic window” where exceeding the dose levels could be hazardous, for example, acetaminophen.

For example, active ingredients susceptible to misuse may include all opioid agonists or mixed agonist-antagonists, partial agonists, including but not limited to alfentanil, allylprodine, alphaprodine, anileridine, benzylmorphine, bezitramide, butorphanol, clonitazene, codeine, desomorphine, diamorphone, dihydrocodeine, dihydromorphine, dipipanone, eptazocine, ethoheptazine, ethylmorphine, fentanyl, hydrocodone, hydromorphone, isomethadone, ketobemidone, levorphanol, lofentanil, meperidine, meptazinol, metazocine, methadone, morphine, narceine, nicomorphine, norlevorphanol, normethadone, nalorphine, nalbuphene, normorphine, opium, oxycodone, oxymorphone, papaveretum, pentazocine, phenazocine, propheptazine, properidine, pro-poxyphene, sufentanil, tilidine, tramadol, mixtures of any of the foregoing, salts of any of the foregoing, and the like.

Further active ingredients susceptible to misuse that could be combined with active ingredients as described herein further include but are not limited to: acetaminophen, albendazole, alclometasone dipropionate, alprazolam, alprostadil, amiloride, aminosalicylate, aminosalicylic acid, amitriptyline hydrochloride, ammonium chloride, amobarbital, amodiaquine hydrochloride, amoxapine, amphetamine sulfate, acetylsalicylic acid, apomorphine, apraclonidine, aspirin, anipamil, azaperone, azathioprine, beclomethasone diproionate, bendroflumethiazide, benzonatate, betaine, betamethasone, betaxolol, betanechol chloride, biotin, biperiden, botulism antitoxin, bromocriptine mesylate, bumetanide, bupivacaine, busulfan butabarbital sodium, butalbital, combinations of butalbital, caffeine and aspirin and codeine, beta-carotene, calcifediol, calcium carbonate, calcium citrate, calcium salts, candicidin, captopril, carbachol, carbamazepine, carbidopa, carboprost tromethamine, carisoprodol, casanthranol, cascara, chlorambucil, chloramphenicol, chlordiazepoxide, chloroquine phosphate, chlormadinone acetate, chlorothiazide, chloroxylenol, chlorpromazin, chlorpropamide, chlorthalidone, chlorzoxazone, cholecalciferol, chromic chloride, cimetidine, cinoxazin, cisplatin, clindamycin hydrochloride, -palmitate and -phosphate, clioquinol, clofazimine, clofibrate, clomriphene citrate, clonazepam, cinnarizine, clonidine hydrochloride, clorsulon, clotrimazole, cyanocobalamin, coccidioidin, codeine, colchicine, corticotropin, corisone acetate, cyclophosphamide, cyclosporine, cysteine hydrochloride, danazol, dapsone, dehydrocholic acid, demeclocycline, desipramine, desoximetasone, desoxycorticosterone acetate, dexamethasone, dexpanthenol, dextroamphetamine, dextromethorphan, diazepam, diazoxide, dibucaine, dichlorphenamide, dienestrol, diethylpropion hydrochlorid, diethylstilbestrol, diflunisal, digitalis, dicoumarol, digitoxin, digoxin, dihydroergotamine, dihydrostreptomycin, dihydrotachysterol, dihydroxyaluminium amino acetate, dihydroxyaluminium sodium carbonate, diltiazem hydrochloride, dimercaprol, dipyridamole, disopyramide phosphate, dobutamine hydrochloride, dopamine hydrochloride, doxepin hydrochloride, doxycycline, doxycycline hyclate, doxylamine cuccinate, dronabinol, droperidol, drotaverine, dydrogesterone, dyphylline, guaifenesin, enalapril maleate, analaprilat, ephedrine, epinephrine, equilin, ergocalciferol, ergoloid mesylates, ergonovine maleate, ergotamine tartrate, erythrityl tetranitrate, erythromycin, estradiol, estriol, estrogene, estrone, ethambutol hydrochloride, ethinyl estradiol, ethionamide, ethopropazine hydrochloride, ethotoin, etoposide, famotidine, fenoprofen, ferrous fumatate, ferrous gluconate, ferrous sulfate, flucytosine, fludrocortisone acetate, flunisolide, fluocinolone acetonide, fluocinonide, fluorescein sodium, fluorometolone, fluorouracil, fluoxymesterone, fluphenazine, flurandrenolide, flurazpam, flurbiprofen, folic acid, furazolidone, flunitrazepam, furosemide, gemfibrozil, gentamicin, gentian violet, glutarate, glutethimide, chorionic gonadotropin, gramicidin, griseofulvin, guaifenesin, guanabenz, guanadrelsulfate, halazone, haloperidol, haloprogin, halothane, heparin calcium, hexylresorcinol, histamine phosphate, histoplasmin, hydrochlorothiazide, hydrocodone bitartrate, hydrocortisone, hexobarbital, hydroflumethiazide, hydromorphone hydrochloride, hydroquinone, hydroxocobalamin, hydroxyamphetamine, hydroxychloroquine sulfate, hydroxyprogesterone caproate, hydroxyurea, hydroxine hydrochloride, hydroxine pamoate, ibuprofen, ifosfamide, imipramide, imipramide hydrochloride, indapamide, indomethacin, insulin, inulin, iocetamid, iodoquinol, iohexol, iopamidol, ipodate calcium, ipodate sodium, isocarboxacid, isoetharine hydrochloride, isoflurane, isoniacid, isopropamide iodine, isoproterenol hydrochloride, isosorbide dinitrate, isotretenoin, isoxsuprine hydrochloride, ketoprofen, ketoconazole, labetalol hydrochloride, leucine, leucovorin calcium, levamisole hydrochloride, levocarnithine, levodopa, levonorgestrel, levorphanol tartrate, levothyroxine sodium, lidocaine, lincomycin hydrochloride, lindane, liothyronine sodium, liotrix, lisinopril, lithium carbonate, loperamide hydrochloride, loracarbef, lonetil, lorazepam, lovastatin, loxapine, lysine, mafenide acetate, magaldrte, malathinon, manganese salts, manganese, maprotiline hydrochloride, mazindol, measle virus vaccine, mebendazole, mebrofenin, mecamylamine hydrochloride, meclocycline, meclofenamate sodium, medroxyprogesterone acetate, mefenamic acid, megestrol acetate, meglumine, melphalan, menadiol sodium diphosphate, menadione, menotropine, meperidine, mephenytoin, mephobarbital, meprednisone, meprobamate, mercaptopurine, mesoridazine besylate, mestranol, metaproterenol sulfate, metaraminol bitartrate, methacycline hydrochloride, methadone hydrochloride, methamphetamine hydrochloride, methazolamide, methdilazine, methenamine, methicillin sodium, methimazole, methionine, methocarbamol, methotrexate, methoxsalen, methoxyflurane, methsuximide, methyclothiazide, methylbenzethonium chloride, methyldopa, methylergonovine maleate, methylphenidate hydrochloride, methylprednisolone, methyltestosterone, methysergide maleate, metoclopramide, metolazone, meoprolol tartrate, metronidazole, metyrapone, metyrosine, mexiletine hydrochloride, mexiletine hydrochloride, miconazole, minocycline hydrochloride, minoxidil, mitomycin, mitotane, molindone hydrochloride, monobenzone, morphine sulfate, mupirocin, medazepam, mefruside, methandrostenolone, methylsulfadiazine, nadolol, nafcillin, nafcillin sodium, nalidixic acid, nalorphine, naloxone, nandrolone decanoate, nandrolone phenpropionate, naproxen, natamycin, neomycin, neomycin sulfate, neostimine bromide, nitrofurantoin, nalidixic acid, nifedipine, nitrazepam, nitrofurantoin, nitromerson, nizatidine, nonoxynol 9, norethindrone, norethindrone acetate, norfloxacin, norgestrel, nortriptyline hydrochloride, noscapine, novobiocin sodium, nystatin, opium, oxacillin sodium, oxamniquine, oxandrolone, oxazepam, oxprenolol hydrochloride, oxtriphylline, oxybenzone, oxybutynin chloride, oxycodone hydrochloride, oxycodone, oxymetazoline hydrochloride, oxymetholone, oxymorphone hydrochloride, oxyphenbutazone, oxytetracycline, padimate, panreatin, pancrelipase, papain, panthenol, papaverin hydrochloride, parachlorophenol, paramethasone acetate, paregoric, paromomycin sulfate, pentaerythritol tetranitrate, pentazocine, pentazocine salts, pentobarbital sodium, perphenazine, pertussis, phenacemide, phenazopyridine hydrochloride, phendimetrazine tartrate, phenelzine sulfate, phenmetrazine hydrochloride, phenobarbital, phenoxybenzamine hydrochloride, phentermine hydrochloride, phenylalanine, phenylbutazone, phenylephrine hydrochloride, phenylpropanolamine hydrochloride, physostigmine, phytonadione, pilocarpine, pimozide, pindolol, piperazine, piroxicam plicamycin, poliovirus vaccine inactivated, polycarbophil, polymycin b sulfate, polythiazide, potassium chloride, potassium citrate, potassium cluconate, potassium iodine, potassium sodium tartrate, povidone iodine, pralidoxime chloride, pramoxine hydrochloride, pramezam, prazepam, praziquantel, prazosin hydrochloride, prazosin hydrochloride, prednisolone, prilocalne, primaquine, primidone, probenecid, probucol, procainamide hydrochiorid, procaine hydrochloride, procarbacine hydrochloride, prochlorperazine, prochlorperazine maleate, progesterone, proline, promazine, promazine hydrochloride, promazine, promethazine, promethazine hydrochloride, propafenone hydrochloride, proparacaine hydrochloride, propoxycaine hydrochloride, propoxyphene hydrochloride, propoxyphene napsylate, propanolol hydrochloride, propyliodone, propylthiouracil, propylthiouracil, protriptyline hydrochloride, pseudoephedrine hydrochloride, pyrantel pamoate, pyrazinamide, pyrethrum extract, pyridostigmine bromide, pyridoxine hydrochloride, pyrimethamnine, pyroxylin, pyrvinium pamoate, phenacetin, phenytoin, prednisone, uinidine gluconate, quinidine sulfate, rabies vaccine, racepinephrine ranitidine, rauwolfia serpentina, resorcinol, ribavirin, riboflavin, rifampin, ritodrine, rubella virus vaccine, saccharin, saccharin sodium, salicylamide, salicylic acid, salsalata, secobarbital sodium, selenius acid, selenium sulfate, sennasenme, simethicone, sodium ascorbate, sodium bicarbonate, sodium fluoride, sodium gluconate, sodium iodide, sodium lactate, sodium nitrite, sodium ditroprusside, sodium salicylate, spironolactone, stannozolol, streptomycin, sucralfate, sulfacetamide, sulfadiazine, reserpine, sulfadioxine, sulfamerazine, sulfamethazine, sulfamethizole, sulfamethoxazole, sulfamethoxydiazine, sulfapyridin, sulfasalazine, sulfaperin, sulfathiazole, sulfisoxazole, sulfinpyrazone, sulindac, suprofen, stilains, tamoxifen citrate, temacepam, terbutaline sulfate, terfenadine, terpin, testolacton, testosterone, tolazamide, tolbutamide, tetracaine, tetracycline, tetrahydrocycline, theophylline, thiabendazole, thiamine hydrochloride, thiamin, thiamylal, thiethylperazine thimerosal, thioguanine, thioridazine hydrochloride, thistrepton, thiotepa, thiothixene, threonine, thyroid, ticarcillin, timolol, tioconazole, titaniumdioxide, tolazamide, tolbutamide, tolmetin, tolnaftate, trazodone hydrochloride, tretinoin, triacetin, triamcinolone, triamterene, triazolam, trichorfon, trichlormethiazide, trientine hydrochloride, trifluoperazine hydrochloride, triflupromazine, trihexyphenidyl hydrochloride, trimeprazine tartrate, trimethadione, trimethobenzamide hydrochloride, trimethoprim, trioxsalen, tripelennamine, triprolidine, trisulfapyrimidine, tropicamide, trypsin, tryptohan, tuberculin, tyloxapol, tyropanoate sodium, tyrosine, tyrothricin, thyrothricin bethamethasone, thiotic acid, sotalol, salbutamol, norfenefiine, silymarin, dihydroergotamine, buflomedil, etofibrate, indometacin, urea, valine, valproic acid, vancomycin hydrochloride, vasopressin, verapramil, vidarabine, vinblastine, vincristine, vitamins, warfarin, yellow fever vaccine, zinc acetate, zinc carbonate, zinc chloride, zinc gluconate, beta acetyl digoxin, piroxicam, haloperidol, ISMN, amitriptylin, diclofenac, nifedipine, verapamil, pyritinol, nitrendipin, doxycycline, bromhexine, methylprdnisolone, clonidine, fenofibrate, allopurinol, pirenyepine, levothyroxin, tamoxifen, metildigoxin, o-(beta-hydroxyethyl)rutoside, propicillin, aciclovir mononitrate, paracetamol, naftidrofuryl, pentoxiflline, propafenone, acebutolol, L-thyroxin, tramadol, bromocriptine, loperamide, ketotifen, fenoterol, cadobelisate, propanolol, enalaprilhydrogen maleate, bezafebrate, ISDN, gallopamil, xantinol nicotinate, digitoxin, flunitrazepam, bencyclane, dexapanthenol, pindolol, lorazepam, diltiazem, piracetam, phenoxymethylpenicillin, furosemide, bromazepam, flunarizin, erythromycin, metoclopramide, acemetacin, ranitidin, biperiden, metamizole, doxepin, dipotassium chloroazepate, tetrazepam, estramustine phosphate, terbutaline, captopril, maprotiline, prazosin, atenolol, glibenclamide, cefaclor, etilfrine, cimetidine, theophylline, hydromorphone, ibuprofen, pnimidone, clobazam, oxaceprol, medroxyprogesterone, flecainid, pyridoxal 5 phosphate glutaminate, hymechromone, etofylline clofibrate, vincamine, cinnarizine, diazepam, ketoprofen, flupentixol, molsimine, glibornuride, dimetinden, melperone, soquinolol, dihydrocodeine, clomethiazole, clemastine, glisoxepide, kallidinogenase, oxyfedrine, baclofen, carboxymethylcysteine, thioridazine, betahistine, L-tryptophan, murtol, bromelaine, prenylamine, salazosulfapyridine, astemizol, sulpiride, benzerazide, dibenzepine, acetylsalicylic acid, miconazol, nystatin, ketoconazole, sodium picosulfate, coltyramine, gemfibrocil, rifampicin, fluocortolone, mexiletin, amoxicillin, terfenadrin, mucopolysaccharide polysulfade, triazolam, mianserin, tiaprofenic acid, amezinium metilsulfate, mefloquine, probucol, quinidine, carbamazepine, L-aspartate, penbutolol, piretanide, aescin amitriptyline, cyproterone, sodium valproinate, mebeverine, 5-aminosalicylic acid, dihydralazine, magaldrate, phenprocoumon, amantadine, naproxen, carteolol, famotidine, methyldopa, auranofine, estriol, nadolol, levomepromazine, doxorubicin, medofenoxate, azathioprine, flutamide, norfloxacin, fendiline, prajmalium bitartrate, lipid derivatives of phosphonatides, amphiphilic polymers, adenosine derivatives, sulfated tannins, monoclonal antibodies, and metal complexes of water soluble texathyrin.

A therapeutic dose of an active ingredient is an amount of active ingredient recommended on a label.

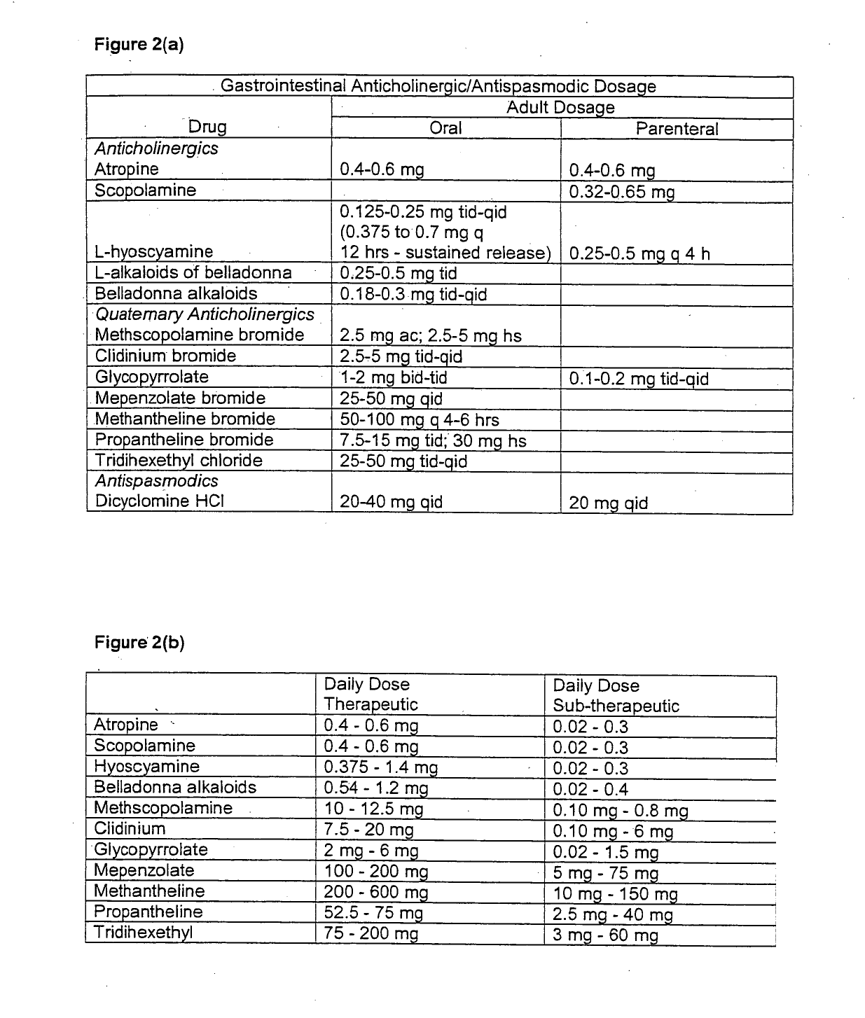

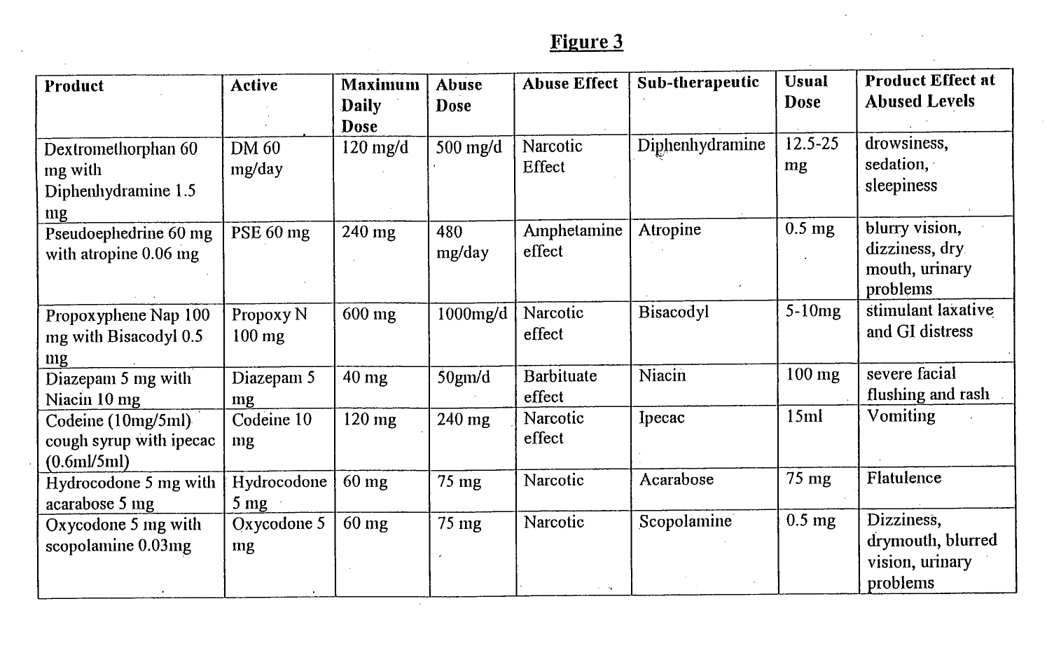

A sub-therapeutic dose of an anticholinergic compound or a compound capable of eliciting an undesirable response is an amount less than that sufficient to elicit an undesirable response. For example, a sub-therapeutic dose may be 1/10th, ⅕th or ½ the amount necessary to elicit an undesirable response. FIG. 2 includes examples of sub-therapeutic doses for various compounds capable of eliciting an undesired response. With respect to anticholinergics capable of eliciting an undesirable CNS response, the anticholinergic may be present in a ratio of 30,000:1 to 250:1 by weight of active ingredient to anticholinergic.

A therapeutically effective amount of an active ingredient susceptible to misuse may range from about 0.001 to 30 mg/kg body weight, about 0.01 to 25 mg/kg body weight, about 0.1 to 20 mg/kg body weight, and or about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to, the severity of the disease or condition, disorder, or disease, current or previous treatments, the general health and/or age of the subject, and other diseases present. Moreover, treatment of a subject with a therapeutically effective amount of the compounds can include a single treatment or can include a series of treatments. In an example, a subject is treated with the compound in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of the compound used for treatment may increase or decrease over the course of a particular treatment. Changes in dosage may result and become apparent from the results of diagnostic assays as described herein.

The pharmaceutical compositions may be formulated in conventional manner using one or more physiologically acceptable carriers or excipients. They may take many forms: oral solid, including tablets and capsules, injectable, oral liquid, intranasal, transdermal, powders, rapid dissolve oral formulations, lozenges, or formulated for pulmonary delivery. Compositions may also be formulated for nasal or dry powder inhaler delivery.

For oral administration, the pharmaceutical compositions may take the form of, for example, liquids, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound as well as immediate and delayed release.

For buccal administration the compositions may take the form of tablets or lozenges formulated in conventional manner.

For administration by inhalation, the compounds may be conveniently delivered in the form of an aerosol spray presentation from pressurized packs or a nebulizer, dry powder, inhalation, aqueous nasal solutions or suspensions. A suitable propellant may be used, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. A pack may take of the form of a substrate with a 1-10 day dosing regimen deposited thereon with two or more tablets or capsules taken at each dosing interval. One or more tablets or capsules at each dosing may include the active ingredient and one or more tablets or capsules taken at each dosing include the sub-therapeutic dose of a compound capable of eliciting an undesirable response. The tablets or capsules taken at each dosing interval are indistinguishable to the patient.

The following non-limiting examples are included to merely demonstrate a few formulations that could be used.

### EXAMPLE 1

### (Prophetic) Acetaminophen/Pyridium

500 mg acetaminophen may be combined with 40 mg pyridiium (phenazopyridine) in a tablet when it is desired that urine color change be exhibited when a patient takes more than 8 tablets per day. This combination provides for a change in urine color when a patient ingests an amount of acetaminophen exceeding 4 grams per day.

### EXAMPLE 2

### (Prophetic) Dextromethorphan/Pyridium

30 mg of dextromethorphan may be combined with 40 mg pyridium in a tablet when it is desired that urine color change be exhibited when a patient takes more than 8 tablets per day. 60 mg of dextromethorphan may be combined with 80 mg of pyridium in a tablet when a patient takes more than 4 tablets per day. This combination provides for a change in urine color when a patient ingests an amount of acetaminophen exceeding 240 mg per day.

### EXAMPLE 3

### (Prophetic) Oxycodone/Methylene Blue

5 mg oxycodone may be combined with 50 mg methylene blue when it is desired that urine color change occur in patients taking more than 6 tablets per day. This combination provides for a change in urine color when a patient ingests an amount of acetaminophen exceeding 30 mg per day.

### EXAMPLE 4

### (Prophetic) Diazepam/Niacin Combination Product

Diazepam may be combined with a sub-therapeutic dose of Niacin. An example includes a tablet with 5 mg diazepam and 10 mg Niacin. A patient consuming more than 8 tablets in excess of the maximum recommended dosage of 8 tablets per day would experience severe facial flushing and rash.

### EXAMPLE 5

### (Prophetic) Hydrocone/Acarbose Combination Product

Hydrocodone may be combined with a sub-therapeutic dose of acarbose. An example includes a tablet with 5 mg hydrocodone and 5 mg acarbose. A patient consuming more than 12 tablets in excess of the maximum recommended dosage of 12 tablets per day would experience flatulance.

### EXAMPLE 6

### (Prophetic) Oxycodone/Scopolamine Combination Product

Oxycodone may be combined with a sub-therapeutic dose of scopolamine. An example includes a tablet with 5 mg oxycodone and 0.03 mg scopolamine. A patient consuming more than 12 tablets in excess of the maximum recommended dosage of 12 tablets per day would experience dizziness, blurry vision, and dry mouth.

### EXAMPLE 7

### (Prophetic) Codeine/Ipecac Combination Product

Codeine may be combined with a sub-therapeutic dose of ipecac. An example includes a syrup with 10 mg per 5 ml codeine and 0.6 per 5 ml ipecac. A patient consuming more than the maximum recommended would experience vomiting.

### EXAMPLE 8

### (Prophetic) Pseudoephedrine/Atropine Combination Product

Pseudoephedrine may be combined with a sub-therapeutic dose of atropine. An example includes a tablet with 60 mg pseudoephedrine and 0.06 mg atropine. A patient consuming more than 4 tablets in excess of the maximum recommended dosage of 4 tablets per day would experience dizziness, blurry vision, and dry mouth.

### EXAMPLE 9

### (Prophetic) Pronoxyyphene Nap/Bisacodyl Combination Product

Propoxyphene may be combined with a sub-therapeutic dose of Bisacodyl. An example includes a tablet with 100 mg hydrocodone and 0.5 mg bisacodyl. A patient consuming more than 6 tablets in excess of the maximum recommended dosage of 6 tablets per day would experience stimulant laxative and gastro intestinal distress.

### EXAMPLE 10

### (Prophetic) Dextromethorphan/Diphenhydramine Combination Product

Dextromethorphan may be combined with a sub-therapeutic dose of diphenhydramine. An example includes a tablet with 60 mg dextromethorphan and 1.5 mg diphenhydramine. A patient consuming more than 8 tablets in excess of the maximum recommended dosage of 2 tablets per day would experience drowsiness and sedation.

### EXAMPLE 11

### (Prophetic) Hydrocodone/Atropine Combination Product

Hydrocodone may be combined with a sub-therapeutic dose of atropine. An example includes a tablet with 5 mg hydrocodone and 0.02 mg atropine. A patient consuming more than 15 tablets in excess of the maximum recommended dosage of 12 tablets per day would experience dizziness, blurry vision, and dry mouth.

### EXAMPLE 12

### (Prophetic) Dextromethorphan/Atropine Combination Product

Dextromethorphan may be combined with a sub-therapeutic dose of atropine. An example includes a tablet with 60 mg dextromethorphan and 0.06 mg atropine. A patient consuming more than 8 tablets in excess of the maximum recommended dosage of 2 tablets per day would experience dizziness, blurry vision and dry mouth.

What is claimed is:

1. A multiple dose pharmaceutical dosage form comprising an effective amount of an active ingredient for a patient in need of treatment thereof and a urine indicator capable of changing the color of the patient's urine when a desired dosage of the active ingredient is exceeded by ingestion of multiple doses in excess of a desired treatment regimen.

2. The pharmaceutical composition of claim 1, wherein the indicator is phenazopyridine or pharmaceutically acceptable salts thereof.

3. The pharmaceutical composition of claim 1, wherein the color shift indicator is methylene blue or pharmaceutically acceptable salts thereof.

4. The pharmaceutical composition of claim 1, wherein the indicator is mannosej or pharmaceutically acceptable salts thereof.

5. The pharmaceutical composition of claim 1, wherein the indicator is, melanine, phenolphthalein, phenosulfonphthalein, or pharmaceutically acceptable salts thereof.

6. The pharmaceutical composition of claim 1, wherein the indicator is one or more riboflavins or pharmaceutically acceptable salts thereof.

7. The pharmaceutical composition of claim 1, wherein the indicator is one or more tannins or pharmaceutically acceptable salts thereof.

8. The pharmaceutical dosage form of claim 1, wherein the active ingredient is acetominophen, dextromethorphan, or oxycodone.

9. The pharmaceutical dosage form of claim 1, wherein the urine indicator is present in an amount sufficient to shift color of a patient's urine at least 10 nm in the visible spectrum when a desired total dosage of active ingredient is exceeded.

10. The pharmaceutical dosage form of claim 1, wherein the urine indicator is present in an amount 50 mg to 750 mg.

11. The composition of claim 1, wherein the pharmaceutical dosage form is a tablet, capsule, liquid, or suspension.

12. A method for indicating when a patient ingesting multiple doses of a pharmaceutical dosage form exceeds a desired treatment regimen comprising:
a. observing the color of a patient's urine;
b. ingesting one or more doses of a pharmaceutical dosage form including an effective amount of an active ingredient for a patient in need of treatment thereof and a urine color shift indicator capable of changing the color of the patient's urine when a desired dosage of the active ingredient is exceeded by ingestion of multiple doses in excess of a desired treatment regimen;
c. observing the color of a patient's urine after ingesting one or more of the doses;
d. comparing the color of the patent's urine before and after ingesting one or more of the doses to determine if there is a substantial change in color of the patient's urine.

13. The method of claim 12, further comprising reducing the number of doses ingested by a patient when it is determined that there is a substantial change in the color of the patient's urine before and after administration of one or more doses.

14. The method of claim 12, wherein a substantial change in color of a patient's urine is one that represents a color shift of at least 10 nm.

15. The pharmaceutical composition of claim 12, wherein the indicator is phenazopyridine or pharmaceutically acceptable salts thereof.

16. The pharmaceutical composition of claim 12, wherein the color shift indicator is methylene blue or pharmaceutically acceptable salts thereof.

17. The pharmaceutical composition of claim 12, wherein the indicator is mannose or pharmaceutically acceptable salts thereof.

18. The pharmaceutical composition of claim 12, wherein the indicator is melanine phenolphthalein, phenosulfonphthalein, or pharmaceutically acceptable salts thereof.

19. The pharmaceutical composition of claim 12, wherein the indicator is one or more riboflavins or pharmaceutically acceptable salts thereof.

20. The pharmaceutical composition of claim 12, wherein the indicator is one or more tannins or pharmaceutically acceptable salts thereof.

21. The pharmaceutical dosage form of claim 12, wherein the active ingredient is acetominophen, dextromethorphan, or oxycodone.

22. The pharmaceutical dosage form of claim 12, wherein the urine indicator is present in an amount 50 to 750 mg.

23. The composition of claim 1, wherein the pharmaceutical dosage form is a tablet, capsule, liquid, or suspension.
